(19) United States
(12) Patent Application Publication
(10) Pub. No.: US 2009/0035292 A1
(43) Pub. Date: Feb. 5, 2009

(54) Use of phosphatases to treat neuroblastomas and medulloblastomas

(76) Inventors: John S. Kovach, East Setauket, NY (US); Zhengping Zhuang, Bethesda, MD (US)

Correspondence Address: COOPER & DUNHAM, LLP, 30 Rockefeller Plaza, 20th Floor, NEW YORK, NY, 10112, US

(21) Appl. No.: 12/221,360

(22) Filed: Aug. 1, 2008

Related U.S. Application Data
(60) Provisional application No. 61/063,970, filed on Feb. 6, 2008.
(60) Provisional application No. 60/963,307, filed on Aug. 3, 2007.

(51) Int. Cl. A61K 38/46; A61K 31/496; A61K 31/34; A61K 38/02; A61K 38/12; A61P 35/00; A61K 31/07; A61K 38/15; A61K 31/44; A61K 33/16

(52) U.S. Cl. 424/94.6; 514/255.01; 514/469; 514/352; 424/676; 514/2; 514/9; 514/725

(57) ABSTRACT

Disclosed herein are methods of treating neuroblastomas and medulloblastomas in a subject comprising administering to the subject a phosphatase ligand in an amount effective to treat the subject. Also disclosed herein are method of treating neuroblastomas and medulloblastomas in a subject comprising administering to the subject a histone deacteylase ligand in an amount effective to treat the subject.

This application claims the benefit of U.S. Provisional Application No. 61/063,970, filed Feb. 6, 2008, and U.S. Provisional Application No. 60/963,307, filed Aug. 3, 2007, the contents of each of which are hereby incorporated by reference

Parts of this invention were created in collaboration with the National Institutes of Health. The Government of the Untied States has certain rights in the invention.

Throughout this application, certain publications are referenced. Full citations for these publications may be found immediately preceding the claims. The disclosures of these publications in their entireties are hereby incorporated by reference into this application in order to more fully describe the state of the art to which this invention relates.

### BACKGROUND OF THE INVENTION

Despite medical advances of the past few decades, cancer continues to plague people of all ages. The prevalence of various forms of cancer and lack of effective treatments for many forms is a testament to the problems these diseases present. Of the many cancers still lacking an effective treatment, neuroblastoma and medulloblastoma are some of the most lethal.

Neuroblastoma probaby derives from primitive sympathetic neural precursors. About half of all neuroblastomas arise in the adrenal medulla, and the rest originate in the paraspinal sympathetic ganglia in the chest or abdomen, or in pelvic ganglia. Neuroblastomas account for 7-10% of all childhood cancers and are the most common cancer diagnosed during infancy with the prevalence about one case in 7,000 live births with 700 new cases per year in the United States. This incidence is fairly uniform throughout the world, at least for industrial nations. The median age at diagnosis for neuroblastoma patients is about 18 months, so approximately 40% are diagnosed by one year of age, 75% by four years of age and 98% by ten years of age. Children older than one year with advanced disease have a more serious prognosis with long-term disease-free status in only 30% of patients despite maximum chemotherapy with bone marrow rescue and maintenance treatment with 13-cis-retinoic acid. When the disease occurs in an adolescent or an adult, prognosis is worse than in younger children.

Medulloblastomas are the most common malignant brain tumors of childhood accounting for more than 20% of pediatric brain tumors. They show both neuronal and glial differentiation. Multimodality treatment including surgery, radio- and chemotherapy have greatly improved the survival of this neoplasm, but more than one third of children with medulloblastomas still die within five years of diagnosis. The remaining survivors experience significant toxicities secondary to therapy. Radiation to the brain is an important component of effective treatment, yet administration of effective radiation doses to children three years or younger frequently results in significant impairment of cognitive ability. Thus in young children, development of new chemotherapy regimens that would provide disease control at least until the child reaches age three and may receive appropriate radiation with reduced chance of severe impairment of neurological function are needed. Antitumor agents which confer potential for long-term survival and have limited toxiticities are thus far lacking.

The subject application provides novel methods of treating neuroblastomas and medulloblastomas.

### SUMMARY OF THE INVENTION

The invention disclosed herein provides a method of treating a subject suffering from a neuroblastoma or a medulloblastoma comprising administering to the subject one or more phosphatase ligand, alone or in combination with one or more retinoid receptor ligand, or one or more histone deacetylase ligand, or both, in each case in an amount effective to treat the subject.

The invention disclosed herein provides a method of treating a subject suffering from a neuroblastoma or a medulloblastoma comprising administering to the subject one or more histone deacetylase ligand, alone or in combination with one or more retinoid receptor ligand, or one or more phosphatase ligand, or both, in each case in an amount effective to treat the subject.

### BRIEF DESCRIPTION OF THE FIGURES

FIG. 1. Treatment with Compound 100 inhibits proliferation of neuroblastoma cells. The neuroblastoma cell line, SH-SY5Y, was exposed to Compound 100 for 4 or 7 days at concentrations of 1 μM (squares), 5 μM (triangles), 10 μM (short dashed line), 20 μM (diamonds), 50 μM (long dashed line) or vehicle only (circles).

FIG. 2. Treatment with Compound 100 and ATRA inhibits proliferation of neuroblastoma cells. The neuroblastoma cell line, SH-SY5Y, was exposed to 5 μM Compound 100 (squares), 25 μM ATRA (circles), the combination of 5 μM Compound 100 and 25 μM ATRA (dashed line) or vehicle only (black line) for 4 or 7 days.

FIG. 3. Treatment with a combination of Compound 100, ATRA and valproic acid severely inhibits proliferation of neuroblastoma cells. The neuroblastoma cell line, SH-SY5Y, was exposed to 10 μM Compound 100 (squares), 2.5 mM valproic acid (circles), 10 μM ATRA (triangles), 10 μM Compound 100 and 2.5 mM valproic acid (large dashed line), 2.5 mM valproic acid and 10 μM ATRA (small dashed line), 10 μM Compound 100 and 10 μM ATRA (dashed and dotted line), 10 μM Compound 100, 2.5 mM valproic acid and 10 μM ATRA (black line) or vehicle alone (diamonds) for 3 or seven days.

FIG. 4. Treatment with Compound 100 inhibits proliferation of medulloblastoma cells. The medulloblastoma cell line, DAOY, was exposed to 20 μM Compound 100 (squares), 5 μM Compound 100 (triangles), 1 μM Compound 100 (x's) or vehicle only (diamonds) for 3 days.

FIG. 5. Treatment with ATRA inhibits proliferation of medulloblastoma cells. The medulloblastoma cell line, DAOY, was exposed to 50 μM ATRA (squares), 20 μM ATRA (triangles), 5 μM ATRA (X's) or vehicle only (diamonds) for 3 days.

FIG. 6. Treatment with valproic acid inhibits proliferation of medulloblastoma cells. The medulloblastoma cell line, DAOY, was exposed to 2 mM valproic acid (squares), 1 mM valproic acid (triangles), 0.5 mM valproic acid (X's) or vehicle only (diamonds) for 3 days.

FIG. 7: Treatment with Compound 100 or with Compound 102 inhibits the proliferation of the medulloblastoma cell line DAOY xenograft tumors in SCID mice. DAOY cells (5 million) were implanted subcutaneously in the flank of SCID mice (Day 0). After the xenografts reached a size of ˜130 cubic mm, treatment was instituted with vehicle alone (control), compound 100 (1.5 mg/kg), or compound 102 (1.5 mg/kg) daily intraperitoneally for 21 days (beginning at day 7). Xenograft masses were measured at days 7, 14 and 21 of treatment.

FIG. 8: Treatment with Compound 205 and Compound 205 in combination with ATRA inhibits proliferation of the medulloblastoma cell line DAOY.

FIG. 9: Treatment with Compound 100 or Compound 205 inhibits the proliferation of the neuroblastoma cell line SHSY xenograft in SCID mice. SHSY cells (5 million) were implanted subcutaneously in the flank of SCID mice. After the xenografts reached a size of ˜100 cubic mm, treatment was instituted with vehicle alone (control), compound 100 (1.5 mg/kg) or compound 205 (10 mg/kg) daily intraperitoneally for 14 days. Xenograft masses were measured day 7 and day 14 of treatment.

### DETAILED DESCRIPTION OF THE INVENTION

This invention provides a method of treating a subject suffering from neuroblastomas and medulloblastomas, comprising administering to the subject one or more phosphatase ligand, alone or in combination with one or more retinoid receptor ligand, or one or more histone deacetylase ligand, or both, in each case in amounts effective to treat the subject.

The invention disclosed herein provides a method of treating a subject suffering from a neuroblastoma or a medulloblastoma comprising administering to the subject one or more histone deacetylase ligand, alone or in combination with one or more retinoid receptor ligand, or one or more phosphatase ligand, or both, in each case in an amount effective to treat the subject.

The phosphatase ligand may be selected from the group consisting of 1-nor-okadaone, antimonyl tartrate, bioallethrin, calcineurin, cantharidic acid, cantharidin, calyculin, cypermethrin, DARPP-32, deamidine, deltamethrin, diaminopyrroloquinazolines, endothal, endothal thioanhydride, fenvalerate, fostriecin, imidazoles, ketoconazole, L-4-bromotetramisole, levamisole, 1-p-bromotetramisole, d-p-bromotetramisole, p-hydroxylevamisole, microcystin LA, microcystin LR, microcystin LW, microcystin RR, molybdate salts, okadaic acid, okadol, norcantharidin, pentamidine, pentavalent antimonials, permethrin, phenylarsine oxide, phloridzin, protein phosphatase inhibitor-1 (I-1), protein phosphatase inhibitor-2 (I-2)pyrophosphate, salubrinal, sodium fluoride, sodium orthovanadate, sodium stibogluconate, tartrate salts, tautomycin, tetramisole, thrysiferyl-23-acetate, vanadate, vanadium salts and antileishmaniasis compounds, including suramin and analogues thereof.

In a presently preferred embodiment of the invention, the phosphatase ligand is a protein phosphatase inhibitor, such as endothal thioanhydride, endothal, norcantharidin or okadaic acid. The protein phosphatases of the subject application can be tyrosine-specific, serine/threonine-specific, dual-specificity phosphatases, alkaline phosphatases such as levamisole, and acid phosphatases.

In another embodiment of the invention, the phosphatase ligand is a protein phosphatase inhibitor having the structure

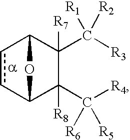

wherein bond α is present or absent; R₁ and R₂ is each independently H, O⁻, OR₉, where R₉ is H, alkyl, alkenyl, alkynyl or aryl, or R₁ and R₂ together are ═O; R₃ and R₄ are each different and each is OH, O—, OR₉, SH, S⁻, SR₉,

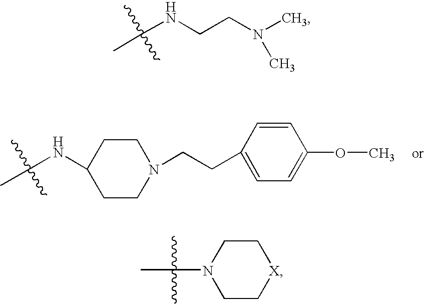

where X is O, S, NR₁₀, N⁺R₁₀R₁₀, where each R₁₀ is independently alkyl, substituted C₂-C₁₂ alkyl, alkenyl, substituted C₄-C₁₂ alkenyl, alkynyl, substituted alkynl, aryl, substituted aryl where the substituent is other than chloro when R₁ and R₂ are ═O,

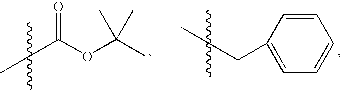

—CH₂CN, —CH₂CO₂R₁₁, —CH₂COR₁₁, —NHR₁₁, —NH⁺(R₁₁)₂, wherein each R₁₁ is independently alkyl, alkenyl or alkynyl, each of which is substituted or unsubstituted, or H;
R₅ and R₆ is each independently H, OH, or R₅ and R₆ taken together are ═O; and R₇ and R₈ is each independently H, F, Cl, Br, SO₂Ph, CO₂CH₃, CN, COR₁₂, or SR₁₂, where R₁₂ is H, aryl or a substituted or unsubstituted alkyl, alkenyl or alkynyl, or a salt, enantiomer or zwitterion of the compound.

In another embodiment, the protein phosphatase inhibitor described above has the structure

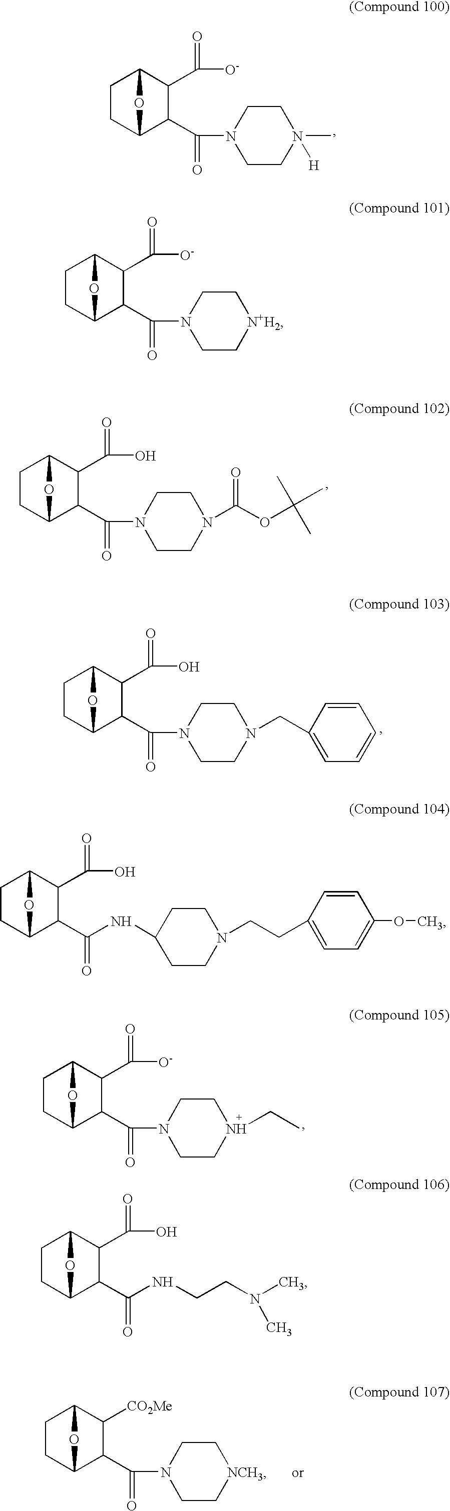

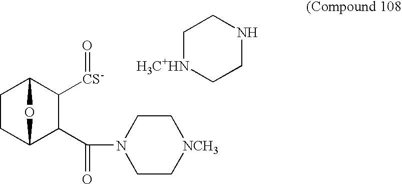

The above identified compounds, Compounds 100-108, can be obtained by methods described in PCT International Application No. PCT/US08/01549.

In the method of the invention, the histone deacetylase ligand may be an inhibitor, e.g. the histone deacetylase inhibitor HDAC-3 (histone deacetylase-3). The histone deacetylase ligand may also be selected from the group consisting of 2-amino-8-oxo-9,10-epoxy-decanoyl, 3-(4-aroyl-1H-pyrrol-2-yl)-N-hydroxy-2-propenamide, APHA Compound 8, apicidin, arginine butyrate, butyric acid, depsipeptide, depudecin, HDAC-3, m-carboxycinnamic acid bis-hydroxamide, N-(2-aminophenyl)-4-[N-(pyridin-3-ylmethoxycarbonyl)aminomethyl]benzamide, MS 275, oxamfiatin, phenylbutyrate, pyroxamide, scriptaid, sirtinol, sodium butyrate, suberic bishydroxamic acid, suberoylanilide hydroxamic acid, trichostatin A, trapoxin A, trapoxin B and valproic acid. In the preferred embodiment of the invention, the inhibitor is valproic acid.

In another embodiment, the HDAC inhibitor is a compound having the structure

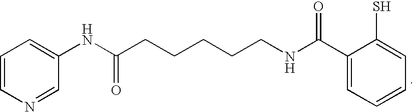

In the method of the invention, the retinoid receptor ligand may be a retinoid, such as a retinoic acid, e.g. cis retinoic acid or trans retinoic acid. The cis retinoic acid may be 13-cis retinoic acid and the trans retinoic acid may be all-trans retinoic acid. In the preferred embodiment, the retinoic acid is all-trans retinoic acid (ATRA).

Retinoid receptor ligands used in the method of the invention include vitamin A (retinol) and all its natural and synthetic derivatives (retinoids).

In the method of the invention, the retinoid receptor ligand may be selected from the group consisting of b,g-selective 6-(5,6,7,8-tetrahydro-5,5,8,8-tetramethyl-2-naphthalenyl)-2-naph-thalenecarboxylic acid (TTNN), Z-oxime of 6-(5,6,7,8-tetrahydro-5,5,8,8-tetramethyl-2-naphthalenylcarbonyl)-2-naphthalenecarboxylic acid (SR11254), 4-(5,6,7,8-tetrahydro-5,5,8,8-tetramethyl-2-anthracenyl)benzoic acid (TTAB), 4-[1-(5,6,7,8-tetrahydro-5,5,8,8-tetramethyl-2-naphthalenyl)-cyclopropyl]benzoic acid (SR11246), 4-[1-(5,6,7,8-tetrahydro-3,5,5,8,8-pentamethyl-2-naphthalenyl)-2-methylpropenyl]benzoic acid (SR11345), and 2-(6-carboxy-2-naphthalenyl)-2-(5,6,7,8-tetrahydro-5,5,8,8-tetramethyl-2-naphthalenyl)-1,3-dithiolane (SR11253).

In an embodiment of any of the methods disclosed herein, the subject is a mammal.

### Terms

As used in this application each of the following terms has the meaning set forth below.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

The compounds described in the present invention are in racemic form or as individual enantiomers. The enantiomers can be separated using known techniques, such as those described, for example, in Pure and Applied Chemistry 69, 1469-1474, (1997) IUPAC.

As used herein, “zwitterion” means a compound that is electrically neutral but carries formal positive and negative charges on different atoms. Zwitterions are polar, have high solubility in water and have poor solubility in most organic solvents.

The compounds disclosed herein may also form zwitterions. For example, a compound having the structure

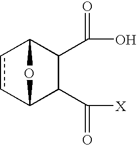

may also for the following zwitterionic structure

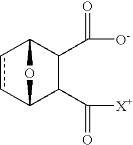

where X is as defined throughout the disclosures herein.

Certain embodiments of the disclosed compounds can contain a basic functional group, such as amino or alkylamino, and are thus capable of forming pharmaceutically acceptable salts with pharmaceutically acceptable acids, or contain an acidic functional group and are thus capable of forming pharmaceutically acceptable salts with bases. The instant compounds therefore may be in a salt form. As used herein, a “salt” is a salt of the instant compounds which has been modified by making acid or base salts of the compounds. The salt may be pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. For a description of possible salts, see, e.g., Berge et al. (1977) “Pharmaceutical Salts”, J. Pharm. Sci. 66:1-19.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement, and specifically includes methyl, ethyl, propyl, butyl, pentyl, hexyl, and so on. An embodiment can be C₁-C₁₂ alkyl. “Alkoxy” represents an alkyl group as described above attached through an oxygen bridge. “Hydroxyalkyl” represents an alkyl group as described aboved with a hydroxyl group. Hydroxyalky groups include, for example, (CH₂)₁₋₁₀OH and includes CH₂OH, CH₂CH₂OH, CH₂CH₂CH₂OH and so forth.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2, . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2, . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, which includes F, Cl, Br, and I, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

As used herein, “therapeutically effective amount” means an amount sufficient to treat a subject afflicted with a disease (e.g. neuroblastoma or medulloblastoma) or to alleviate a symptom or a complication associated with the disease.

As used herein, “treating” means slowing, stopping or reversing the progression of a disease, particularly neuroblastoma and medulloblastoma.

The subject application encompasses compounds which inhibit the enzyme histone deacetylase (HDAC). These HDAC enzymes posttranslationally modify histones (U.S. Patent Publication No. 2004/0197888, Armour et al.) Histones are groups of proteins which associate with DNA in eukaryotic cells to form compacted structures called chromatin. This compaction allows an enormous amount of DNA to be located within the nucleus of a eukaryotic cell, but the compact structure of chromatin restricts the access of transcription factors to the DNA. Acetylation of the histones decreases the compaction of the chromatin allowing transcription factors to bind to the DNA. Deacetylation, catalysed by histone deacetylases (HDACs), increases the compaction of chromatin, thereby reducing transcription factor accessibility to DNA. Therefore, inhibitors of histone deacetylases prevent the compaction of chromatin, allowing transcription factors to bind to DNA and increase expression of the genes.

The human neuroblastoma cell line SH-SY5Y is available from the European Collection of Cell Cultures, Health Protection Agency, Porton Down SP40JG Salisbury, Wiltshire UK, as ECACC No. 94030304.

The human medulloblastoma cell line DAOY is available from the American Type Culture Collection (ATCC), P.O. Box 1549, Manassas, Va., 20108, as ATCC No. HTB-186.

All combinations of the various elements described herein are within the scope of the invention.

The following Experimental Details are set forth to aid in an understanding of the subject matter of this disclosure, but are not intended to, and should not be construed to, limit in any way the claims which follow thereafter.

### EXPERIMENTAL DETAILS

### Materials and Methods

### Example 1

### Effect of Cantharidin Analogs on SH-SY5Y Cells

The cantharidin homolog that was evaluated was the Compound 100, which was obtained from Lixte Biotechnology Holdings, Inc., 248 Route 25A, No. 2, East Setauket, N.Y., which has the structure:

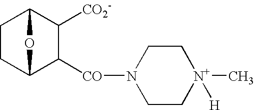

Another cantharidin homolog that was evaluated was the compound Compound 102, which was obtained from Lixte Biotechnology Holdings, Inc., 248 Route 25A, No. 2, East Setauket, N.Y., which has the structure:

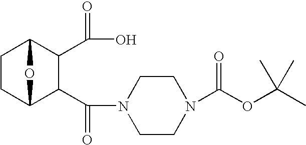

### In Vitro Experiments:

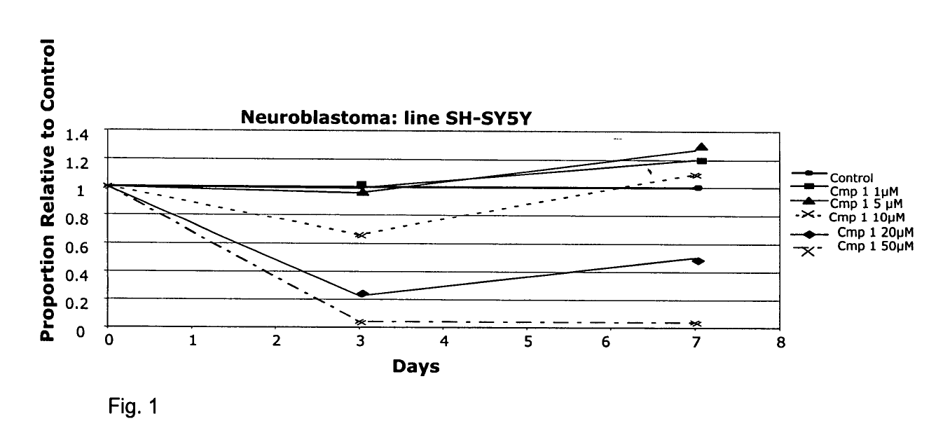

The neuroblastoma cell line, SHSY5Y, was exposed to the cantharidin analog Compound 100 for 4 or 7 days at concentrations of 1, 5, 10, 20 and 50 μM. At the two lower doses, 1 μM and 5 μM, there was little or no inhibition of cell proliferation at day 4 and enhanced cell growth by day 7 as compared to cells exposed to vehicle (media) alone (FIG. 1.) Dose dependent inhibition was observed at day 4 at the three higher doses with escape of growth inhibition for doses less than 50 μM by day 7. At low doses, Compound 100, like several other known protein phosphatase inhibitors, slightly stimulates cellular proliferation (Yi et al., 1988 and Wang, 1989)

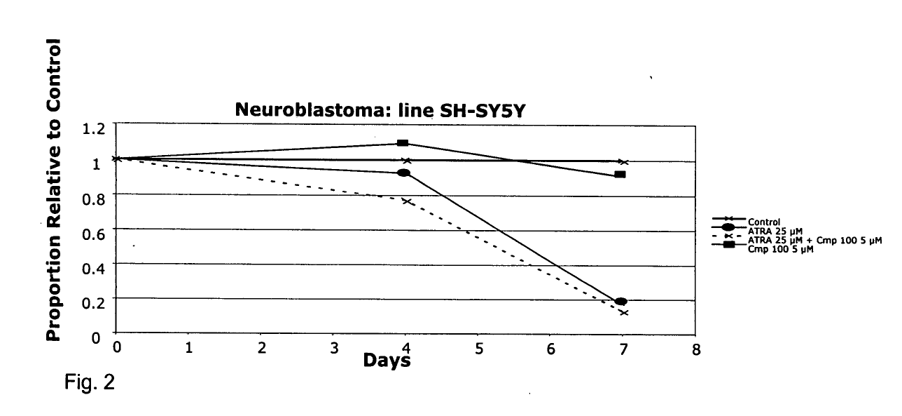

It has been demonstrated previously that although long-term treatment of SH-SY5Y cells with all-trans retinoic acid (ATRA) can inhibit cellular proliferation, short-term treatment of neuroblastomas with ATRA is not sufficient prevent proliferation. In fact, short term treatment (1-3 days) of SH-SY5Y cells with ATRA induced cellular migration and invasion (Joshi et al 2006). However, research performed on human glioblastoma multiforme, an unrelated cancer of the central nervous system, indicated that the combination of ATRA with Compound 100 was highly effective in preventing cellular proliferation (PCT International Application No. PCT/US2007/003095). Subsequently, SH-SY5Y cells were exposed to Compound 100 at a concentration of 5 μM, ATRA at 25 μM or the combination of the two drugs at the aforementioned doses for 4 or 7 days. Compound 100 treatment failed to significantly inhibit cellular proliferation (FIG. 2, squares) while treatment with ATRA did not impair cellular proliferation until after day 4 (FIG. 2, circles). When administered in combination, compound 100 potentiated the extent of inhibition by ATRA at both the day 4 and day 7 timepoints (FIG. 2, dashed line).

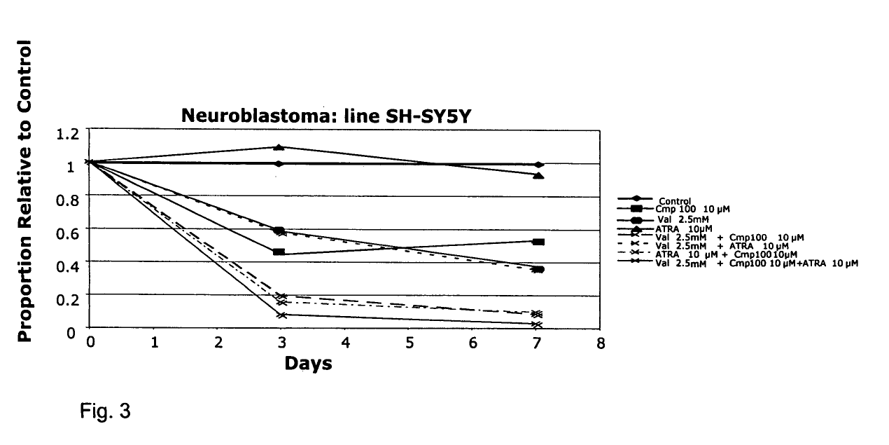

Additionally, previous work has demonstrated that Compound 100 in combination with trichostatin A or valproic acid, two histone deacetylase inhibitors with different mechanisms of action, inhibits several types of human cancer cells in vitro, including glioblastoma multiforme, better then would be expected from the combination of the agents alone. (PCT International Application No. PCT/US2007/003095). Consequently, the same neuroblastoma cell line, SH-SY5Y was exposed to different combinations of compounds and the effects on cellular proliferation evaluated. Treatment with 10 μM ATRA did not prevent proliferation at day 4 (FIG. 3, triangles), consistent with previous results, while proliferation was greatly reduced in cells treated with either 10 μM Compound 100 or 2.5 mM valproic acid (FIG. 3, squares and circles, respectively). Cells treated with 2.5 mM valproic acid and 10 μM ATRA exhibited inhibition of proliferation (FIG. 3, short dashed line); however the cells treated with 2.5 mM valproic acid and 10 μM Compound 100 (FIG. 3, long dashed line), 10 μM ATRA and 10 μM Compound 100 (FIG. 3, dashed-dotted line) or 2.5 mM valproic acid, 10 μM ATRA and 10 μM Compound 100 (FIG. 3, black line) exhibited high levels of proliferation inhibition, indicating that Compound 100 synergistically enhanced the activity of ATRA, valproic acid and valproic acid in combination with ATRA.

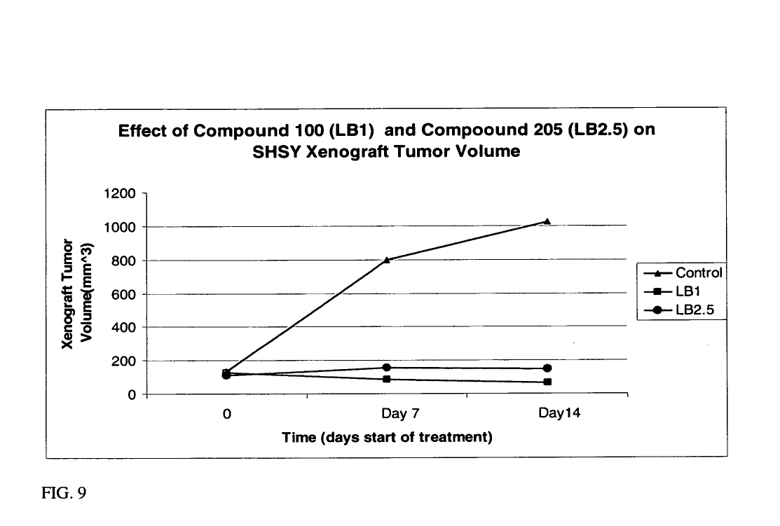

It is also demonstrated that Compound 100 inhibits the proliferation of the neuroblastoma cell line SHSY when SHSY cells are implanted in SCID mice (FIG. 9).

### Discussion

Because cure of neuroblastoma in intermediate and high-risk patients is not assured, there is a need for improved methods of treatment. For example, high-risk patients usually receive aggressive chemotherapy with very high doses of drugs following surgery, and then by high dose chemotherapy with bone marrow rescue and, at times, total body irradiation (Berthold et al., 2005). Of potential relevance to the discoveries described herein, is the fact that at least some neuroblastomas are sensitive to retinoids. When 13-cis-retinoic acid is given for 6 months to high risk patients who have been through highly aggressive chemotherapeutic, surgical and radiation treatments, survival is improved significantly (Matthay et al., 1999). The fact that Compound 100 activity with ATRA is better than would be expected either compound 100 alone or ATRA alone makes it reasonable that the use of compound 100 in combination with a retinoid would be effective against neuroblastoma.

### Example 2

### Effect of Cantharidin Analogs on DAOY Cells

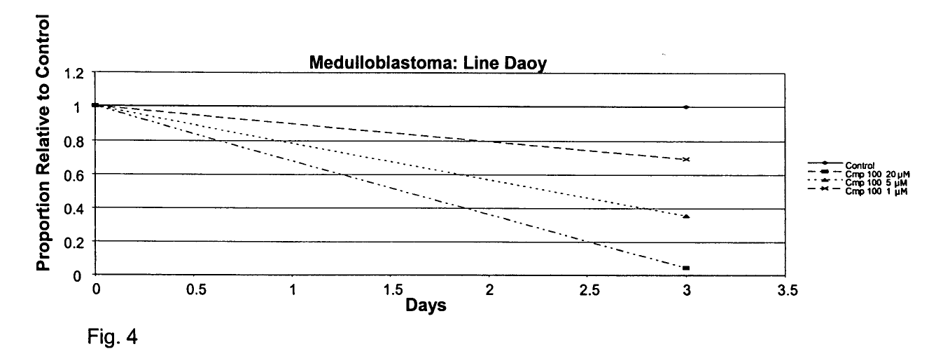

The medulloblastoma cell line, DAOY, was exposed to the cantharidin analog, Compound 100 at concentrations of 1 μM, 5 μM and 20 μM and evaluated for cellular proliferation over the course of three days. DAOY cells treated with vehicle only (media) exhibited no change in cellular proliferation while the DAOY cells treated with Compound 100 all had decreased rates of cellular proliferation as compared to the control, with the cells treated with 20 μM Compound 100 exhibiting the greatest decrease in cellular proliferation (FIG. 4, squares). Therefore, Compound 100 even at low concentration is capable of preventing cellular proliferation.

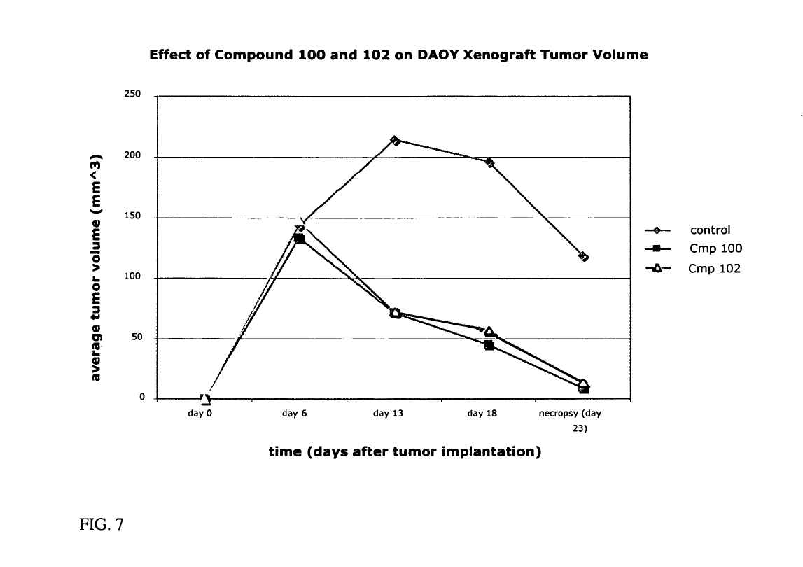

It is also demonstrated the Compound 100 and Compound 102 both inhibit the proliferation of DAOY cells when implanted subcutaneously in SCID mice (FIG. 7).

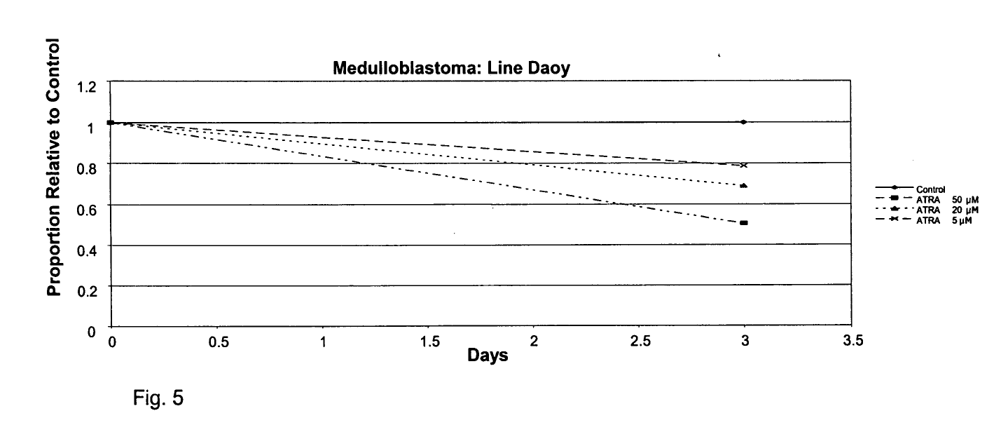

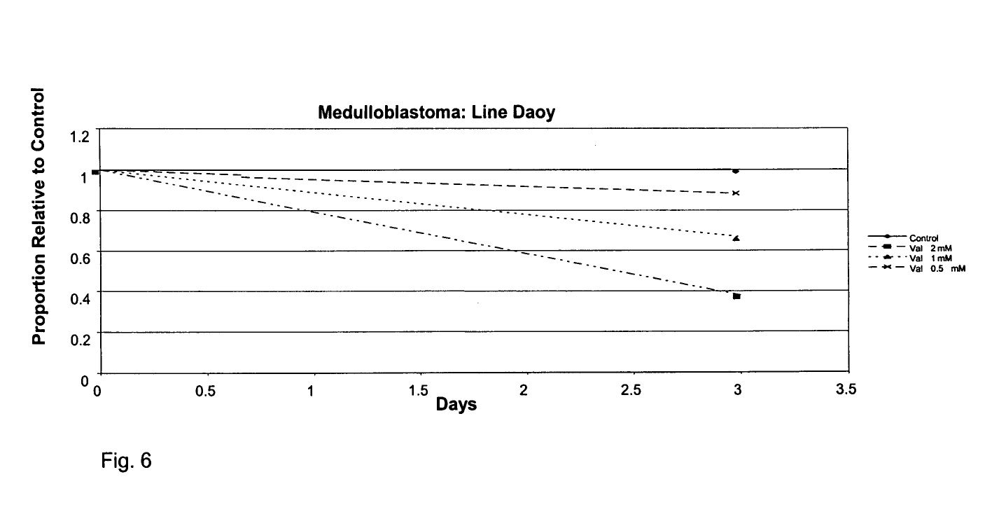

Recent studies have reported that treating DAOY cells with varying concentration of all-trans retinoic acid (ATRA) inhibits cellular proliferation (Chang et al., 2007; Gumireddy et al., 2003). To confirm these observations, cultured DAOY cells were treated with 50 μM, 20 μM, 5 μM or vehicle only for three days and examined for cellular proliferation. As expected, cells treated with vehicle only showed no inhibition of cellular proliferation while all three concentrations of ATRA inhibited proliferation to varying degrees (FIG. 5). Likewise, a recent report indicated that the well-tolerated anticonvulsant and histine deacetylase inhibitor, valproic acid, suppressed cell proliferation in 10 days in DAOY cells exposed to 1 mmol/L valproic acid or 21 days to 0.6 mmol/L valproic acid (Li, et al., 2005). In these studies, however, inhibition of proliferation of DAOY cells treated with 2 mM, 1 mM or 0.5 mM valproic acid was observed over the course of three days (FIG. 6), indicating that these medulloblastoma cells highly sensitive to lower concentrations of valproic acid even at early timepoints. Consequently, because it has been determined that proliferation of medulloblastoma cells is inhibited by Compound 100, ATRA and valproic acid as single agent, it is reasonable to expect that the combination of compound 100 with each of these compounds or a regimen of all three agents may be effective in the treatment of medulloblastoma.

### Example 3

### Effect of HDAC Inhibitors on DAOY Cells

The HDAC inhibitor that was evaluated was the Compound 205, which was obtained from Lixte Biotechnology Holdings, Inc., 248 Route 25A, No. 2, East Setauket, N.Y., which has the structure:

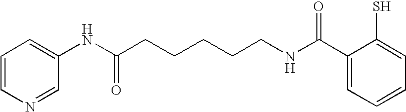

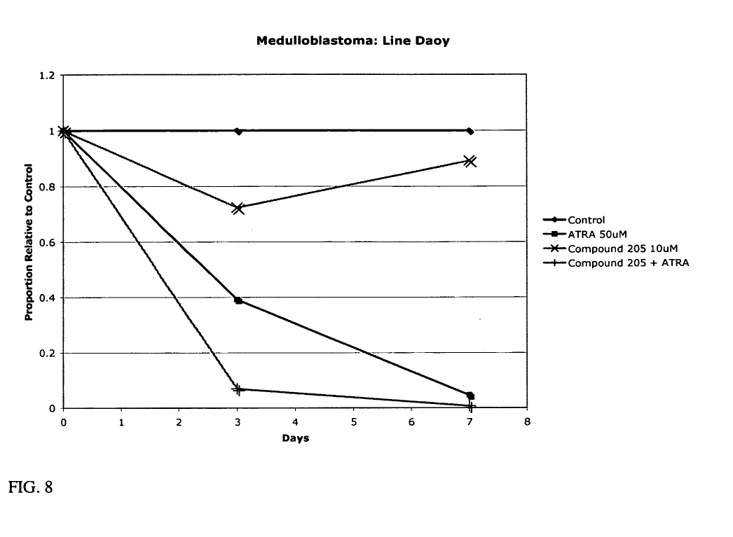

The medulloblastoma cell line, DAOY, was exposed to the HDAC inhibitor, Compound 205 at 10 μM, ATRA at 50 μM, and the compound 205 at 10 μM combined with ATRA at 50 μM, and evaluated for cellular proliferation over the course of seven days. DAOY cells treated with vehicle only (media) exhibited no change in cellular proliferation while the DAOY cells treated with Compound 205 alone and ATRA alone all had decreased rates of cellular proliferation as compared to the control. DAOY cells treated with compound 205 in combination with ATRA, however, had a marked decrease in the rate of cellular proliferation. (FIG. 8) Therefore, we have shown that Compound 205 is active against medulloblastoma cell line DAOY. We have also shown the Compound 205 in combination with ATRA is synergistically active against medulloblastoma cell line DAOY.

### Example 4

### Effect of HDAC Inhibitors on SHSY Cells

It is also shown that treatment with Compound 100 and Compound 205 inhibits the proliferation of the neuroblastoma cell line SHSY implanted in SCID mice. SHSY cells (5 million) were implanted subcutaneously in the flank of SCID mice. After the xenografts reached a size of ˜100 cubic mm, treatment was instituted with vehicle alone (control), Compound 100 (1.5 mg/kg) or Compound 205 (10 mg/kg) daily intraperitoneally for 14 days. Xenograft masses were measured day 7 and day 14 of treatment. As shown in FIG. 9, treatment with Compound 205 inhibited the proliferation of the neruoblastoma cell line SHSY implanted in SCID mice.

### REFERENCES

1. U.S. Patent Application No. 2004/0197888, Armour et al.
2. PCT International Application No. PCT/US2007/003095
3. PCT International Application NO. PCT/US2008/01549
3. Berthold, F., et al. Lancet Oncol. (2005) 6:649-658
4. Chang, Q., et al. J. Neurooncol (2007) 84:263-267
5. Gumireddy, K., et al. Clinical Cancer Research (2003) 9:4065-4059
6. Joshi, S., et al. Oncogene (2006) 25:240-274
7. Li, X-N., et al. Mol Cancer Ther. (2005) 4(12):1912-1922
8. Matthay. K K., et al. N. Engl. J Med. (1999) 341:1165-1173
9. Wang, G-S., et al. Journal of Ethnopharmacology (1989) 26:147-162
10. Yi, S-N., et al. Bulletin of Hunan Medical University (1988) 13:327

1. A method of treating a subject suffering from a neuroblastoma or a medulloblastoma comprising administering to the subject a phosphatase ligand in an amount effective to treat the subject.

2. A method of treating a subject suffering from a neuroblastoma or a medulloblastoma comprising administering to the subject a histone deacetylase ligand in an amount effective to treat the subject.

3. The method of claim 1 further comprising administering to the subject a retinoid receptor ligand in an amount such that the amount of each of the phosphatase ligand and the retinoid receptor ligand is effective to treat the subject.

4. The method of claim 1 further comprising administering to the subject a histone deacetylase ligand in an amount such that the amount of each phosphatase ligand and the histone deacetylase ligand is effective to treat the subject.

5. The method of claim 1 further comprising administering to the subject both a retinoid receptor ligand and a histone deacetylase ligand each in an amount such that the amount of each of the phosphatase ligand, the histone deacetylase ligand and the retinoid receptor ligand is effective to treat the subject.

6. The method of claim 1, wherein the phosphatase ligand is a protein phosphatase inhibitor.

7. The method of claim 1, wherein the phosphatase ligand is selected from the group consisting of 1-nor-okadaone, antimonyl tartrate, bioallethrin, calcineurin, cantharidic acid, cantharidin, calyculin, cypermethrin, DARPP-32, deamidine, deltamethrin, diaminopyrroloquinazolines, endothal, endothal thioanhydride, fenvalerate, fostriecin, imidazoles, ketoconazole, L-4-bromotetramisole, levamisole, microcystin LA, microcystin LR, microcystin LW, microcystin RR, molybdate salts, okadaic acid, okadol, norcantharidin, pentamidine, pentavalent antimonials, permethrin, phenylarsine oxide, phloridzin, protein phosphatase inhibitor-1 (I-1), protein phosphatase inhibitor-2 (I-2) pyrophosphate, salubrinal, sodium fluoride, sodium orthovanadate, sodium stibogluconate, tartrate salts, tautomycin, tetramisole, thrysiferyl-23-acetate, vanadate, vanadium salts and antileishmaniasis compounds, including suramin and analogues thereof.

8. The method of claim 1, wherein the phosphatase ligand has the structure 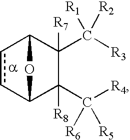
wherein
bond α is present or absent;
R₁ and R₂ is each independently H, O⁻, OR₉,
where R₉ is H, alkyl, alkenyl, alkynyl or aryl,
or R₁ and R₂ together are ═O;
R₃ and R₄ are each different and each is OH, O⁻, OR₉, SH, S—, SR₉ 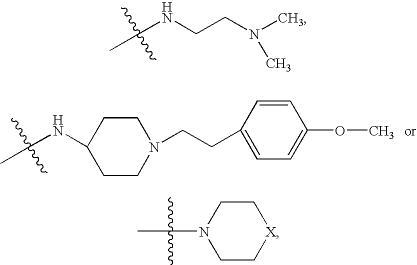
where X is O, S, NR₁₀, N⁺R₁₀R₁₀,
where each R₁₀ is independently alkyl, substituted C₂-C₁₂ alkyl, alkenyl, substituted C₄-C₁₂ alkenyl, alkynyl, substituted alkynl, aryl, substituted aryl where the substituent is other than chloro when R₁ and R₂ are ═O, 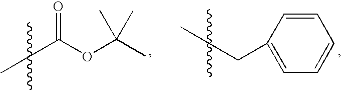
—CH₂CN, —CH₂CO₂R₁₁, —CH₂COR₁₁, —NHR₁₁, —NH⁺(R₁₁)₂
wherein each R₁₁ is independently alkyl, alkenyl or alkynyl, each of which is substituted or unsubstituted, or H;
R₅ and R₆ is each independently H, OH,
or R₅ and R₆ taken together are ═O; and
R₇ and R₈ is each independently H, F, Cl, Br, SO₂Ph, CO₂CH₃, CN, COR₁₂, or SR₁₂,
where R₁₂ is H, aryl or a substituted or unsubstituted alkyl, alkenyl or alkynyl,
or a salt, enantiomer or zwitterion of the compound.

9. The method of claim 2, wherein the histone deacetylase ligand is an inhibitor.

10. The method of claim 9, wherein the inhibitor is valproic acid.

11. The method of claim 10, wherein the inhibitor has the structure

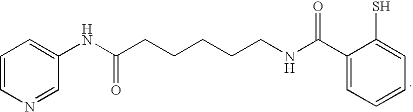

12. The method of claim 2, wherein the histone deacetylase ligand is selected from the group consisting of 2-amino-8-oxo-9,10-epoxy-decanoyl, 3-(4-aroyl-1H-pyrrol-2-yl)-N-hydroxy-2-propenamide, APHA Compound 8, apicidi, arginine butyrate, butyric acid, depsipeptide, depudecin, HDAC-3, m-carboxycinnamic acid bis-hydroxamide, N-(2-aminophenyl)-4-[N-(pyridine-3-ylmethoxycarbonyl)aminomethyl]benzamide, MS 275, oxamfiatin, phenylbutyrate, pyroxamide, scriptaid, sirtinol, sodium butyrate, suberic bishydroxamic acid, suberoylanilide hydroxamic acid, trichostatin A, trapoxin A and trapoxin B.

13. The method of claim 3, wherein the retinoid receptor ligand is a retinoic acid.

14. The method of claim 11, wherein the retinoic acid is all-trans retinoic acid (ATRA).

15. The method of claim 1, wherein the subject is a mammal.

16. (canceled)
